(19) United States
(12) Patent Application Publication
(10) Pub. No.: US 2007/0073083 A1
(43) Pub. Date: Mar. 29, 2007

(54) Process for the production of olefins

(76) Inventor: John Glenn Sunley, Yorkshire (GB)

Correspondence Address: NIXON & VANDERHYE, PC, 901 NORTH GLEBE ROAD, 11TH FLOOR, ARLINGTON, VA, 22203, US

(21) Appl. No.: 10/556,783

(22) PCT Filed: May 18, 2004
(86) PCT No.: PCT/GB04/02130

§ 371 Date: Nov. 14, 2005

(30) Foreign Application Priority Data
May 22, 2003 (GB) 0311774.4

(51) Int. Cl. C07C 51/12

(52) U.S. Cl. 562/519

(57) ABSTRACT

A process for the production of olefins from methane comprising partially combusting a mixture of methane, hydrogen and oxygen in contact with a catalyst capable of supporting combustion beyond the normal fuel rich limit of flammability where they are reacted to form a product comprising one or more olefin(s), there being present in the mixture contacted with said catalyst capable of supporting combustion beyond the normal fuel rich limit of flammability less than 20 mol %, based on the total hydrocarbons present, of hydrocarbons other than methane.

The present invention relates to a process for the production of olefins from methane. More particularly to process in which the methane is treated to autothermal cracking.

Autothermal cracking is a process wherein a hydrocarbon containing 2 or more carbon atoms is mixed with oxygen and passed over a catalyst. Combustion is initiated on the catalyst surface and the heat required to raise the reactants to process temperature and to carry out the endothermic cracking process is generated in situ. Such a process is described for example in EP 332289B; EP-529793B; EP-A-0709446 and WO 00/14035.

However, replacing the hydrocarbon containing 2 or more carbon atoms with methane would be advantageous given that methane is a much cheaper feedstock. Furthermore methane has a higher hydrogen to carbon ratio (4:1) than a hydrocarbon containing 2 or more carbon atoms e.g. ethane (3:1) and consequently the production of olefins from methane typically produces more hydrogen.

We have now found that passing methane, oxygen and hydrogen to an autothermal cracker can produce olefins. This is despite the fact that methane would be expected to be significantly less reactive to form olefins than hydrocarbons containing 2 or more carbon atoms, such as ethane and propane.

Accordingly the present invention provides a process for the production of olefins from methane said process comprising partially combusting a mixture of methane, hydrogen and oxygen in contact with a catalyst capable of supporting combustion beyond the normal fuel rich limit of flammability where they are reacted to form a product comprising one or more olefin(s), there being present in the mixture contacted with said catalyst capable of supporting combustion beyond the normal fuel rich limit of flammability less than 20 mol %, based on the total hydrocarbons present, of hydrocarbons other than methane.

The catalyst capable of supporting combustion beyond the fuel rich limit of flammability usually comprises a Group VIII metal as its catalytic component. Suitable Group VIII metals include platinum, palladium, ruthenium, rhodium, osmium and iridium. Rhodium; and more particularly, platinum and palladium are preferred. Typical Group VIII metal loadings range from 0.01 to 100 wt %, preferably, between 0.01 to 20 wt, and more preferably, from 0.01 to 10 wt % based on the total dry weight of the catalyst.

Where a Group VIII catalyst is employed, it is preferably employed in combination with a catalyst promoter. The promoter may be a Group IIIA, IVA, and/or VA metal. Alternatively, the promoter may be a transition metal; the transition metal promoter being a different metal to that which may be employed as the Group VIII transition metal catalytic component.

Preferred Group IIIA metals include Al, Ga, In and TI. Of these, Ga and In are preferred. Preferred Group IVA metals include Ge, Sn and Pb. Of these, Ge and Sn are preferred. The preferred Group VA metal is Sb. The atomic ratio of Group VIII B metal to the Group IIIA, IVA or VA metal may be 1:0.1-50.0, preferably, 1:0.1-12.0.

Suitable metals in the transition metal series include those metals in Group IB to VIII of the Periodic Table. In particular, transition metals selected from Groups IB, IIB, VIB, VIIB and VIII of the Periodic Table are preferred. Examples of such metals include Cr, Mo, W, Fe, Ru, Os, Co, Rh, Ir, Ni, Pt, Cu, Ag, Au, Zn, Cd and Hg. Preferred transition metal promoters are Mo, Rh, Ru, Ir, Pt, Cu and Zn. The atomic ratio of Group VIII metal to transition metal promoter may be 1:0.1-50.0, preferably, 1:0.1-12.0.

Preferably, the catalyst comprises only one promoter; the promoter being selected from Group IIIA, Group IVA, Group VB and the transition metal series. For example, the catalyst may comprise a metal selected from rhodium, platinum and palladium and a promoter selected from the group consisting of Ga, In, Sn, Ge, Ag, Au or Cu. Preferred examples of such catalysts include Pt/Ga, Pt/In, Pt/Sn, Pt/Ge, Pt/Cu, Pd/Sn, Pd/Ge, Pd/Cu and Rh/Sn. The Rh, Pt or Pd may comprise between 0.01 and 5.0 wt %, preferably, between 0.01 and 2.0 wt %, and more preferably, between 0.05 and 1.0 wt % of the total weight of the catalyst. The atomic ratio of Rh, Pt or Pd to the Group IIIA, IVA or transition metal promoter may be 1:0.1-50.0, preferably, 1:0.1-12.0. For example, atomic ratios of Rh, Pt or Pd to Sn may be 1:0.1 to 50, preferably, 1:0.1-12.0, more preferably, 1:0.2-3.0 and most preferably, 1:0.5-1.5. Atomic ratios of Pt or Pd to Ge, on the other hand, may be 1:0.1 to 50, preferably, 1:0.1-12.0, and more preferably, 1:0.5-8.0. Atomic ratios of Pt or Pd to Cu may be 1:0.1-3.0, preferably, 1:0.2-2.0, and more preferably, 1:0.5-1.5.

Alternatively, the promoter may comprise at least two metals selected from Group IIIA, Group IVA and the transition metal series. For example, where the catalyst comprises platinum, the platinum may be promoted with two metals from the transition metal series, for example, palladium and copper. Such Pt/Pd/Cu catalysts may comprise palladium in an amount of 0.01 to 5 wt %, preferably, 0.01 to 2 wt %, and more preferably, 0.01 to 1 wt % based on the total weight of the dry catalyst. The atomic ratio of Pt to Pd may be 1:0.1-10.0, preferably, 1:0.5-8.0, and more preferably, 1:1.0-5.0. The atomic ratio of platinum to copper is preferably 1:0.1-3.0, more preferably, 1:0.2-2.0, and most preferably, 1:0.5-1.5.

Where the catalyst comprises platinum, it may alternatively be promoted with one transition metal, and another metal selected from Group IIIA or Group IVA of the periodic table. In such catalysts, palladium may be present in an amount of 0.01 to 5 wt %, preferably, 0.01 to 2.0 wt %, and more preferably, 0.05-1.0 wt % based on the total weight of the catalyst. The atomic ratio of Pt to Pd may be 1:0.1-10.0, preferably, 1:0.5-8.0, and more preferably, 1:1.0-5.0. The atomic ratio of Pt to the Group IIA or IVA metal may be 1:0.1-60, preferably, 1:0.1-50.0. Preferably, the Group IIIA or IVA metal is Sn or Ge, most preferably, Sn.

For the avoidance of doubt, the Group VIII metal and promoter in the catalyst may be present in any form, for example, as a metal, or in the form of a metal compound, such as an oxide.

The catalyst may be unsupported, such as in the form of a metal gauze, but is preferably supported. Any suitable support may be used such as ceramic or metal supports, but ceramic supports are generally preferred. Where ceramic supports are used, the composition of the ceramic support may be any oxide or combination of oxides that is stable at high temperatures of, for example, between 600° C. and 1200° C. The support material preferably has a low thermal expansion co-efficient, and is resistant to phase separation at high temperatures.

Suitable ceramic supports include corderite, lithium aluminium silicate (LAS), alumina (α-Al₂O₃), yttria stabilised zirconia, alumina titanate, niascon, and calcium zirconyl phosphate. The ceramic supports may be wash-coated, for example, with γ-Al₂O₃.

The catalyst capable of supporting combustion beyond the fuel rich limit of flammability may be prepared by any method known in the art. For example, gel methods and wet-impregnation techniques may be employed. Typically, the support is impregnated with one or more solutions comprising the metals, dried and then calcined in air. The support may be impregnated in one or more steps. Preferably, multiple impregnation steps are employed. The support is preferably dried and calcined between each impregnation, and then subjected to a final calcination, preferably, in air. The calcined support may then be reduced, for example, by heat treatment in a hydrogen atmosphere.

In a preferred embodiment the process involves passing a mixture of methane, hydrogen and oxygen to a catalyst system comprising a catalyst capable of supporting combustion beyond the normal fuel rich limit of flammability and a methane oxidative coupling catalyst.

The catalyst capable of supporting combustion beyond the normal fuel rich limit of flammability may be anyone as herein described above.

The methane oxidative coupling catalyst may be any suitable catalyst and is preferably an inorganic oxide active for coupling. The inorganic oxide may be any reducible multivalent metal oxide, alkali metal oxide or alkaline earth metal oxide. Usually the metal oxide is supported on a basic oxide or a rare earth metal oxide. Advantageously the catalysts may be promoted with the addition of a halide e.g. chloride. Typical methane oxidative coupling catalysts include Li/MgO, Ba/MgO and Sr/La₂O₃.

The catalyst capable of supporting combustion beyond the normal fuel rich limit of flammability and the methane oxidative coupling catalyst may be co-located in the same catalyst bed or alternatively positioned in two sequential beds.

The catalyst capable of supporting combustion beyond the normal fuel rich limit of flammability is preferably positioned upstream of the methane oxidative coupling catalyst with respect to the directional flow of the reactants.

The methane may be passed to the autothermal cracker in admixture with other materials, for example nitrogen, carbon monoxide, carbon dioxide, or steam.

The methane may also be passed to the autothermal cracker in admixture with other hydrocarbons, for example ethane, as long as the mixture of methane, oxygen and hydrogen passed to the autothermal cracker comprises less than 20 mol %, based on the total hydrocarbons present, of hydrocarbons other than methane. Since hydrocarbons other than methane will generally be significantly more reactive than methane itself, these other hydrocarbons will react before the methane, so the inclusion of hydrocarbons other than methane will reduce the conversion of methane. In one extreme, at significant levels of other hydrocarbons, the methane may become essentially inert. Hence, where hydrocarbons other than methane are present, they are present in an amount (in total of said other hydrocarbons) of less than 20 mol % of the total hydrocarbons passed to the autothermal cracker.

Such other hydrocarbons may be present as an impurity in the methane feed. Such other hydrocarbons may also be present in a recycle stream.

Preferably, there is present in the mixture contacted with said catalyst capable of supporting combustion beyond the normal fuel rich limit of flammability less than 10 mol %, based on the total hydrocarbons present, of hydrocarbons other than methane, such as less than 5 mol %, and more preferably less than 2 mol %.

Preferably a feed stream comprising substantially pure methane is passed to the autothermal cracker, for example methane having a purity of at least 99 wt %.

The oxygen may be passed to the autothermal cracker in admixture with other gases e.g. air or diluted with an inert gas example nitrogen. Preferably a feed stream comprising substantially pure oxygen is passed to the autothermal cracker.

Preferably a feed stream comprising substantially pure hydrogen is passed to the autothermal cracker.

The methane, the hydrogen and the oxygen may be passed separately to the autothermal cracker. Usually the oxygen and the methane are pre-mixed prior to passing into the autothermal cracker and contacting the catalyst. Preferably the hydrogen may also be pre-mixed with the oxygen and methane prior to passing into the autothermal cracker.

In the mixture contacted with the catalyst usually the volume ratio of methane to oxygen is between 1:1 and 5:1, such as 1.5:1 and 3:1, more preferably 2:1 and 2.5:1, and usually the volume ratio of hydrogen to oxygen is between 5:1 and 1:1, preferably 1.5:1 and 1:1.

It is preferred, although not essential, to preheat the gases or gas mixtures passing to the autothermal cracker to suitably between 50 to 600° C., preferably between 100-400° C. e.g. 150° C.

The autothermal cracker may suitably be operated at a temperature greater than 800° C. The upper temperature limit may suitably be up to 1100° C., for example up to 1050° C., preferably up to 1000° C. The catalyst exit temperature may suitably be in the range 800° C. to 1100° C., preferably in the range 850° C. to 1050° C. and, most preferably, in the range 900° C. to 1000° C.

The autothermal cracker may be operated at atmospheric or elevated pressure. Pressures of 1 to 40 barg may be suitable, preferably a pressure of 1-5 barg e.g. 1.8 barg is employed. However a total pressure of greater than 5 barg may be used e.g. 10 barg, usually a total pressure of greater than 15 barg. Wherein the autothermal cracking is operated in a pressure range of between 15-40 barg, advantageously between 20-30 barg e.g. 25 barg the need for compression of the product stream to facilitate separation and purification is reduced and preferably eliminated completely.

Preferably the methane and the oxygen are fed to the autothermal cracker in admixture under a Gas Hourly Space Velocity (GHSV) of greater than 70,000 hr⁻. Preferably, the GHSV exceeds 200,000 hr⁻¹, especially greater than 1,000,000 hr⁻¹. For the purposes of the present invention GHSV is defined as: vol. of total feed at normal temperature and pressure (NTP)/Time/(vol. of catalyst bed).

The reaction products are quenched as they emerge from the reaction chamber to avoid further reactions taking place.

Preferably the product stream is quenched by injection of a large volume of nitrogen or steam at a temperature of between 20-600° C. This quench reduces the temperature of the product stream such that the temperature of the stream is reduced from the autothermal cracking temperature of between 800° C. and 1100° C. to a temperature between 750-600° C.

Usually the product stream is cooled to between 750-600° C. within less than 100 milliseconds of formation, preferably within 50 milliseconds of formation and most preferably within 20 milliseconds of formation e.g. within 10 milliseconds of formation.

Wherein the autothermal cracker is operated at a pressure of 15-20 barg usually the products are quenched and the temperature cooled to between 750-600° C. within 20 milliseconds of formation. Advantageously wherein the autothermal cracker is operated at a pressure of greater than 20 barg the products are quenched and the temperature cooled to between 750-600° C. within 10 milliseconds of formation.

The percentage of conversion methane is usually greater than 5%, preferably greater than 10%, and most preferably greater than 20%.

Furthermore the selectivity towards olefins is usually greater than 20%, preferably greater than 30%, and most preferably greater than 40%.

The product stream comprises at least one olefin and may comprise higher olefins, acetylene, dienes, alkanes, hydrogen and carbon dioxide.

The production of carbon monoxide may be advantageous because it can have a greater value than the methane in certain geographical locations.

The invention will now be illustrated using the following examples.

### EXAMPLE 1

A gaseous feed comprising hydrogen, oxygen, methane and nitrogen was pre-heated to a temperature of 130° C. and passed to the autothermal cracker at a GHSV of 750000 hr⁻¹. The gaseous feed had a methane to oxygen ratio of 2.46:1, a hydrogen to oxygen ratio of 2.59:1 and a methane to nitrogen ratio of 7:1. The autothermal cracker contained a catalyst comprising 3% platinum loaded onto an alumina foam and the reaction was carried out at a temperature of 1000° C. and a pressure of 0 barg. The conversion of methane and the product selectivities are shown in Table 1.

TABLE 1

| CH4 | Selectivities % C-mol | | | | | Feed Flows l/min | | | |
|---|---|---|---|---|---|---|---|---|---|
| Conversion | C2H4 | C2H6 | C2H2 | CO | CO2 | methane | hydrogen | oxygen | nitrogen |
| 7.9 | 24.8 | 13.8 | 0.34 | 64.7 | 4.3 | 3.84 | 4.04 | 1.58 | 0.55 |
| 8.1 | 24 | 14.8 | 0.5 | 59.8 | 2.9 | 3.84 | 4.04 | 1.58 | 0.55 |

### EXAMPLE 2

A gaseous feed comprising hydrogen, oxygen, methane and nitrogen was pre-heated to a temperature of 130° C. and passed to the autothermal cracker at a GHSV of 750000 hr⁻¹. The gaseous feed had a methane to oxygen ratio that was varied between 1.77:1 and 2.07:1, a hydrogen to oxygen ratio of 2.02:1 and an oxygen to nitrogen ratio of 3.53:1. The autothermal cracker contained a catalyst comprising 3% platinum loaded onto an alumina foam and the reaction was carried out at a temperature of 1000° C. and a pressure of 0 barg. The conversion of methane and the product selectivities are shown in Table 2.

TABLE 2

| CH4 | Selectivities % C-mol | | | | | Feed Flows l/min | | | |
|---|---|---|---|---|---|---|---|---|---|
| Conversion | C2H4 | C2H6 | C2H2 | CO | CO2 | methane | hydrogen | oxygen | nitrogen |
| 28.7 | 26.1 | 4.3 | 4.2 | 61.9 | 3.6 | 3.63 | 4.14 | 2.05 | 0.58 |
| 19.1 | 30.6 | 9.0 | 0.0 | 57.0 | 3.5 | 3.83 | 4.14 | 2.05 | 0.58 |
| 14.4 | 27.0 | 12.3 | 0.0 | 56.9 | 3.9 | 4.03 | 4.14 | 2.05 | 0.58 |
| 11.2 | 21.8 | 15.0 | 0.0 | 58.8 | 4.4 | 4.24 | 4.14 | 2.05 | 0.58 |

1-13. (canceled)

14. A process for the production of olefins from methane, said process comprising partially combusting a mixture of methane, hydrogen and oxygen in contact with a catalyst capable of supporting combustion beyond the normal fuel rich limit of flammability where they are reacted to form a product comprising one or more olefin(s), there being present in the mixture contacted with said catalyst capable of supporting combustion beyond the normal fuel rich limit of flammability less than 20 mol %, based on the total hydrocarbons present, of hydrocarbons other than methane, wherein in the mixture contacted with the catalyst the volume ratio of hydrogen to oxygen is between 5:1 and 1:1, and wherein the methane and the oxygen are fed to the autothermal cracker in admixture under a Gas Hourly Space Velocity (GHSV) of greater than 70,000 hr⁻¹.

15. The process according to claim 14, wherein the catalyst capable of supporting combustion beyond the fuel rich limit of flammability comprises a Group VIII metal as its catalytic component.

16. The process according to claim 15, wherein the Group VII metal is selected from rhodium, platinum and palladium.

17. The process according to claim 15, wherein the Group VII metal is employed in combination with a catalyst promoter selected from a Group IIIA metal, a Group IVA metal, a Group VA metal and a transition metal, said transition metal promoter being a different metal to that which may be employed as the Group VIII transition metal catalytic component.

18. The process according to claim 14, said process comprising passing a mixture of methane, hydrogen and oxygen to a catalyst system comprising a catalyst capable of supporting combustion beyond the normal fuel rich limit of flammability and a methane oxidative coupling catalyst.

19. The process according to claim 18, wherein the methane oxidative coupling is a reducible multivalent metal oxide, alkali metal oxide or alkaline earth metal oxide.

20. The process according to claim 19, wherein the methane oxidative coupling is promoted with the addition of a halide.

21. The process according to claim 18, wherein the catalyst capable of supporting combustion beyond the normal fuel rich limit of flammability is positioned upstream of the methane oxidative coupling catalyst with respect to the directional flow of the reactants.

22. The process according to claim 14, wherein there is present in the mixture contacted with said catalyst capable of supporting combustion beyond the normal fuel rich limit of flammability less than 10 mol %, based on the total hydrocarbons present, of hydrocarbons other than methane.

23. The process according to claim 22, wherein a feed stream comprising substantially pure methane is passed to the autothermal cracker.

24. The process according to claim 14, wherein, in the mixture contacted with the catalyst, the volume ratio of methane to oxygen is between 1:1 and 5:1.

25. The process according to claim 14, wherein the reaction products are quenched as they emerge from the reaction chamber to cool the product stream to a temperature of between 750-600° C. within less than 100 milliseconds of formation.
